(19) United States
(12) Patent Application Publication
(10) Pub. No.: US 2025/0179402 A1
(43) Pub. Date: Jun. 5, 2025

(54) Cell Cultivation Apparatus

(71) Applicant: SHIMADZU CORPORATION, Kyoto-shi (JP)

(72) Inventors: Tomoki OHKUBO, Kyoto-shi (JP); Chikara MIYAKE, Kyoto-shi (JP)

(21) Appl. No.: 18/843,916

(22) PCT Filed: Mar. 27, 2023
(86) PCT No.: PCT/JP2023/012138

§ 371 Date: Sep. 4, 2024

(30) Foreign Application Priority Data
Mar. 28, 2022 (JP) 2022-052380

(51) Int. Cl. C12M 3/00; C12M 1/00; C12M 1/12; C12M 1/34; C12M 1/36

(52) U.S. Cl. CPC C12M 23/48; C12M 23/38; C12M 29/00; C12M 37/04; C12M 41/40; C12M 41/48

(57) ABSTRACT

A cell cultivation apparatus (100) according to this invention includes a device holder (101), and gas pipes (31) for supplying a gas to cultivation chambers (23). The device holder includes a holder-side lid (10) including an attachment (30) for the gas pipes and holder-side ports (11). The holder-side lid is movable between a connection position and a disconnection position, the connection position being a position in which the gas pipes and the holder-side ports are connected to device-side ports (21) of a device-side lid (25) of a cell cultivation device (20), the disconnection position being a position in which the connection is released.

### TECHNICAL FIELD

The present invention relates to a cell cultivation apparatus, in particular a cell cultivation apparatus for supplying a gas to a cell cultivation device through gas pipes.

### BACKGROUND TECHNOLOGY

Cell cultivation apparatuses for supplying gas to cell cultivation devices through gas pipes are known in the art. Such a technology is disclosed in International Publication No. WO 2016-158233, for example.

International Publication No. WO 2016-158233 discloses a cell cultivation apparatus (Body-on-a-chip unit). According to the above International Publication No. WO 2016-158233, the Body-on-a-chip system is a method in which cultivated cells are used as organ models, and organ models are connected to each other and cultivated, thereby reconstituting phenomena in living organisms relating to organs outside living organisms. The Body-on-a-chip system holds a potentiality as an alternative animal bioassay in which medicinal kinetic analyses that generally require animal bioassay can be carried out in vitro, and particularly, when medicinal kinetics analyses can be evaluated in vitro using cultivated cells originated from human beings, it is expected that data having a favorable correlation with clinical trials can be acquired.

The cell cultivation apparatus in the above International Publication No. WO 2016-158233 includes units each of which is constructed two cultivation chambers in which two cultivation chambers are connected to each other and thus form an organ model as one unit. The cultivation chambers, which forms one unit, are connected by a channel. Cultivation fluids can be circulated in the respective organ model cultivation chambers by sequentially pressurizing the chambers using pneumatic pipes (pressurization lines). In order to circulate the cultivation fluids in the chambers, such gas pipes are connected to a cell cultivation device.

### PRIOR ART

### Patent Document

Patent Document 1: International Publication No. WO 2016-158233

### SUMMARY OF THE INVENTION

### Problems to be Solved by the Invention

In cell cultivation apparatuses such as the cell cultivation apparatus disclosed in the above International Publication No. WO 2016-158233, in order to cultivate cells, addition of a cultivation medium, or the like is required at regular time intervals. Accordingly, when removing the cell cultivation device set in a holder, to open a lid of the cell cultivation device, it is necessary to disconnect all the gas pipes connected. Also, for cell cultivation, it is necessary to connect the gas pipes to the cell cultivation devices. For this reason, it is desired to collectively connect/disconnect the gas pipes for supplying a gas to/from the cultivation devices for cell cultivation.

The present invention is intended to solve the above problem, and one object of the present invention is to provide a cell cultivation apparatus capable of collectively connecting/disconnecting gas pipes for supplying a gas to/from a cultivation device for cell cultivation.

### Means for Solving the Problems

In order to attain the aforementioned object, a cell cultivation apparatus according to one aspect of the present invention includes a device holder for holding a cell cultivation device, the cell cultivation device including cultivation chambers and a device-side lid having device-side ports each of which is connected to corresponding one of the cultivation chambers; and a gas supplier including gas pipes for supplying a gas to the cultivation chambers, wherein the device holder includes a device installation part in which the cell cultivation device is installed, and a holder-side lid arranged above the device installation part, and including an attachment for the gas pipes and holder-side ports, and the holder-side lid is movable between a connection position and a disconnection position, the connection position being a position in which the gas pipes and the holder-side ports are connected to the device-side ports of the device-side lid of the cell cultivation device, the disconnection position being a position in which the gas pipes and the holder-side ports are disconnected from the device-side ports.

### Effect of the Invention

In the cell cultivation apparatus according to one aspect of the present invention, the holder side lid is movable between a connection position and a disconnection position, the connection position being a position in which the gas pipes and the holder-side ports are connected to the device-side ports of the device-side lid of the cell cultivation device, the disconnection position being a position in which the gas pipes and the holder-side ports are disconnected from the device-side ports of the device-side lid of the cell cultivation device.

Accordingly, the gas pipes can be collectively disconnected from the cultivation device by moving the holder-side lid from the connection position to the disconnection position. As a result, it is not necessary to disconnect each of the gas pipes every when the cell cultivation device is removed from the cell cultivation apparatus. Consequently, it is possible to easily remove the cell cultivation device from the device holder for cell cultivation. Also, the gas pipes for supplying a gas can be collectively connected/disconnected to/from the cultivation device by moving the holder-side lid from the disconnection position to the connection position.

### BRIEF DESCRIPTION OF THE DRAWINGS

FIG. 1 is a perspective view showing a cell cultivation apparatus with a device installation part closing a device-side lid in a first position according to one embodiment.

FIG. 2 is a perspective view showing the cell cultivation apparatus with the device installation part in a second position according to the one embodiment.

FIG. 3 is a side view showing the cell cultivation apparatus with the device installation part closing the device-side lid in the first position according to the one embodiment with parts being removed.

FIG. 4 is a side view showing the cell cultivation apparatus with the device installation part closing the device-side lid in the second position according to the one embodiment with parts being removed.

FIG. 5 is a side view showing the cell cultivation apparatus with the device installation part closing the device-side lid in the first position according to the one embodiment.

FIG. 6 is a view showing the cell cultivation apparatus with the device installation part closing the device-side lid in the first position according to the one embodiment as viewed from a top side.

FIG. 7 is a side view showing the cell cultivation apparatus with the device installation part in the second position according to the one embodiment.

FIG. 8 is a side view showing the cell cultivation apparatus with the device installation part in the second position according to the one embodiment from the top side.

FIG. 9 is a schematic diagram view showing the cell cultivation apparatus installed in an incubator in the one embodiment.

FIG. 10 is a schematic diagram illustrating a cell cultivation device according to the one embodiment.

FIG. 11 is a schematic diagram illustrating a cell cultivation device according to a modified example of the one embodiment.

### MODES FOR CARRYING OUT THE INVENTION

Embodiments embodying the present invention will be described with reference to the drawings.

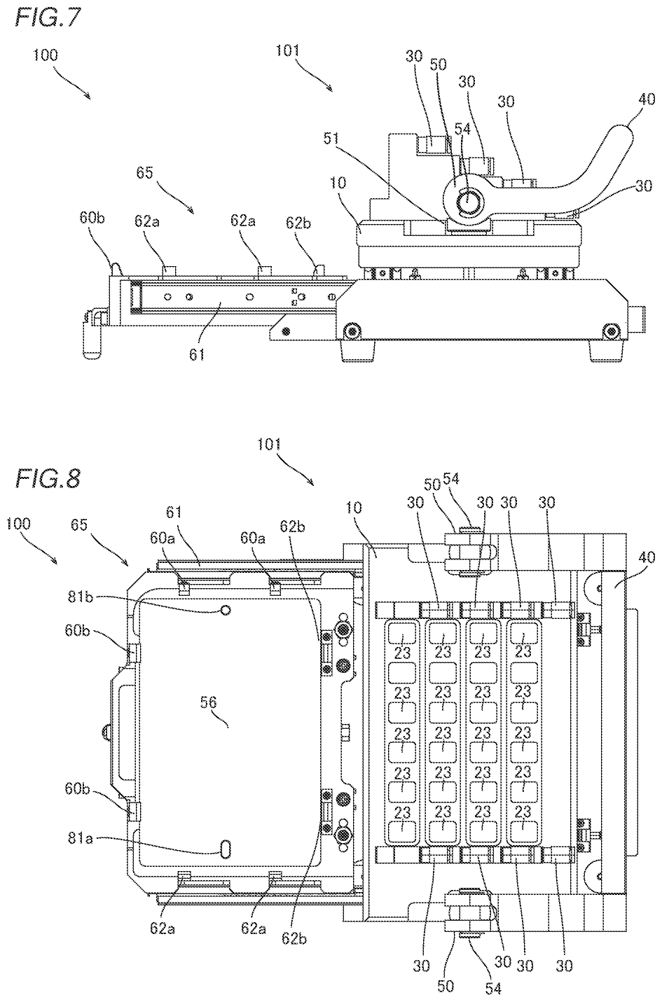

The following description describes a cell cultivation apparatus 100 according to an embodiment with reference to FIGS. 1 to 10.

### Embodiment

### (Structure of Cell Cultivation Apparatus)

The cell cultivation apparatus 100 includes a device holder 101 for holding a cell cultivation device 20, the device holder including cultivation chambers 23 and a device-side lid 25 having device-side ports 21 each of which is connected to corresponding one of the cultivation chambers 23; gas pipes 31; a pneumatic pump 32; a controller 33; and mass flow controllers 34. The mass flow controller 34 is an example of a “pressure control mechanism” in the claims.

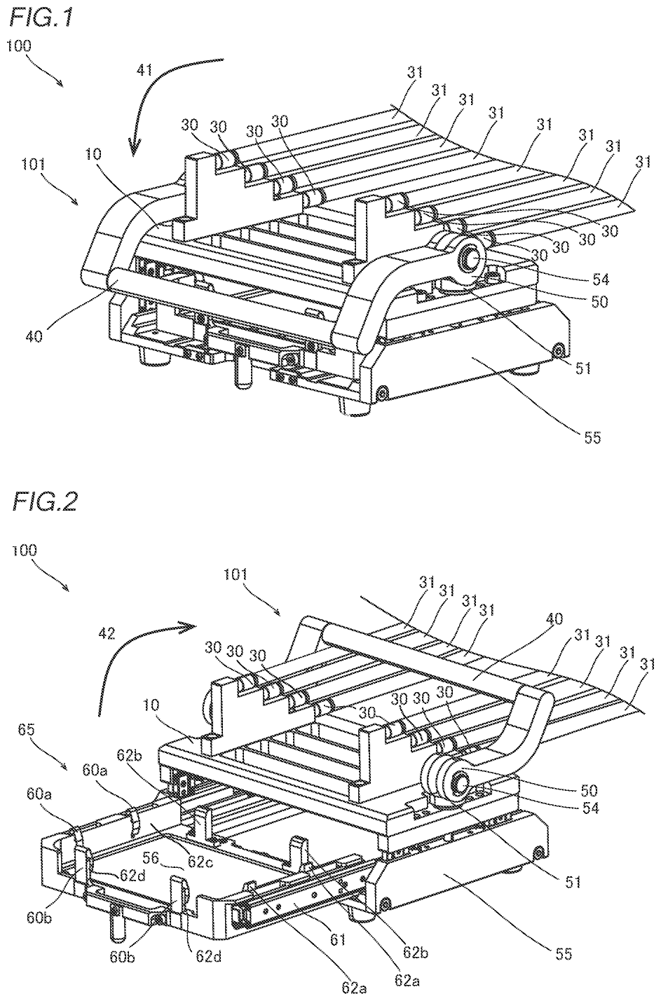

As shown in FIG. 1 to FIG. 8, the device holder 101 includes a holder-side lid 10, an operating lever 40, and a device installation part 65. The device holder 101 is positioned on a top side relative to the device installation part 65 in a height direction. In a case in which the device installation part 65 is positioned in a first position, the holder-side lid 10 is positioned at a position upward of the device installation part 65 in a vertical direction. The operating lever 40 is provided to the holder-side lid 10 (see FIGS. 1 and 2). The operating lever 40 is arranged on a contact plate 51 on the holder-side lid 10, and includes a cam mechanism 50. The operating lever 40 can be moved from a diagonally lower position toward a diagonally upper side. When the operating lever 40 is rotated to a first direction 41, the cam mechanism 50 rotates in a first rotation direction to press down the holder-side lid 10. Also, when the operating lever 40 is rotated to a second direction 42, the cam mechanism 50 rotates in a second rotation direction to raise the holder-side lid 10. Also, the holder-side lid 10 includes attachments 30 for gas pipes 31. The attachments 30 are arranged in a direction extending along an upper surface of the holder-side lid 10, and each of the attachments 30 for the gas pipes 31 is connected to corresponding one of the gas pipes 31.

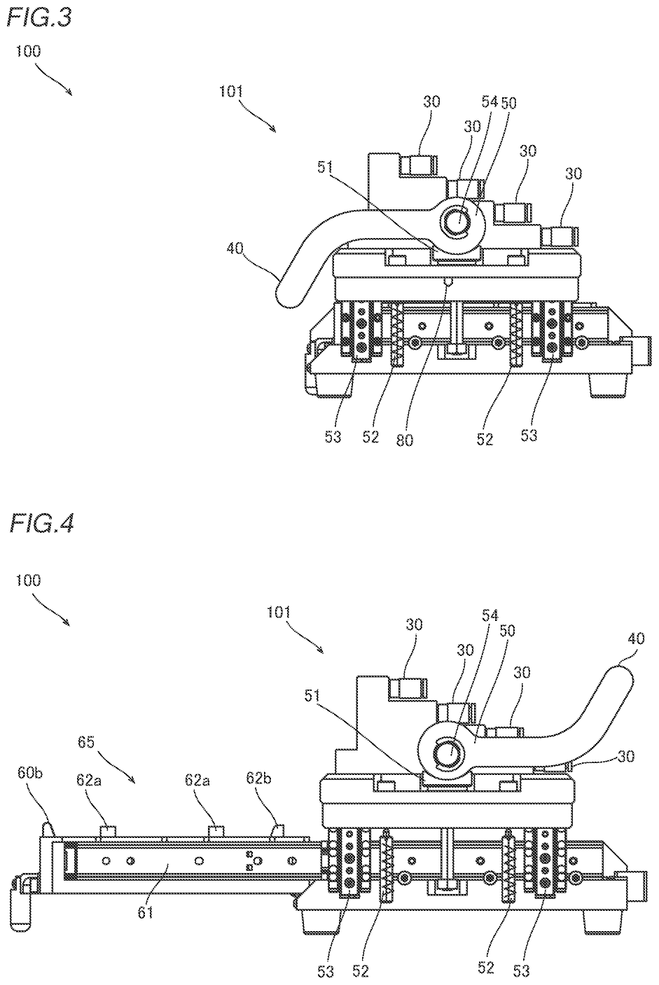

A mechanism for raising/lowering the holder-side lid 10 is now described with reference to FIGS. 3 and 4. The holder-side lid 10 is supported by linear rails 53 and compression coil springs 52. The linear rails 53 and the compression coil springs 52 are arranged in a support 55. The linear rails 53 are arranged provided in both end parts of the holder-side lid 10 in a leftward/rightward direction. The holder-side lid 10 can be linearly moved upward/downward along the linear rails 53. The holder-side lid 10 is supported by the compression coil spring 52 from a bottom side of the holder-side lid, and is biased upward toward a disconnection position, which will be described later. Rotation shafts 54 of the operating lever 40 are arranged in sides of the holder-side lid 10. When the operating lever 40 is rotated in the first rotation direction, the holder-side lid 10 is pressed against the device-side lid 25 by the cam mechanism 50. This is because the rotation of the operating lever 40 in the first rotation direction pushes the contact plate 51 downward relative to a position in which the operating lever is rotated in the second rotation direction. The compression coil springs 52 are examples of a “second biasing element” in the claims.

A tapered pin 80 is arranged on a bottom surface of the holder-side lid 10 as shown in FIG. 3. The pin 80 can engage with a pin hole 81a (see FIG. 8), which will be described later. Because a gap (play) is created between the device and the holder, a horizontal misalignment will occur. The tapered pin 80 can absorb the misalignment. Also, although not illustrated in FIG. 3, a pin for engaging a pin hole 81b (see FIG. 8) is arranged on the bottom surface of the holder-side lid 10.

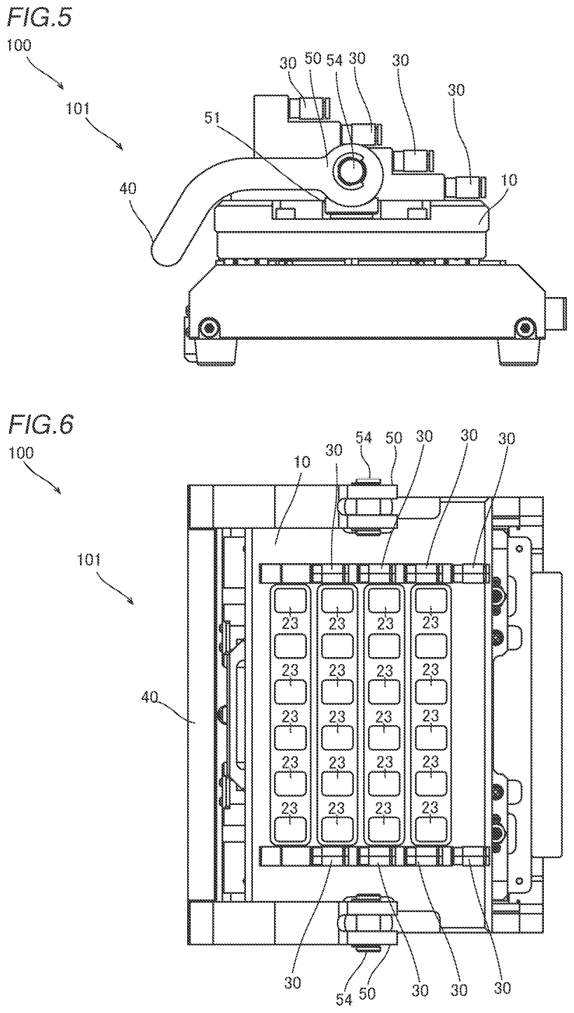

As shown in FIGS. 2, 7 and 8, the device installation part 65 has an installation surface 56 parallel to the device-side lid 25. Also, the device installation part 65 includes the sidewalls arranged around the installation surface 56. Specifically, the sidewalls include a left-side wall 62c and a right-side wall 62a, which face each other in the leftward/rightward direction. Leaf springs 60a arranged on the left-side wall 62c to bias the cell cultivation device against the right-side wall 62a. In addition, the sidewalls include a front-side wall 62d and a rear-side wall 62b, which face each other in a frontward/rearward direction. The front-side wall 62d includes a leaf spring 60b for biasing the cell cultivation device from the front-side wall 62d toward the rear-side wall 62b. Accordingly, the cell cultivation device 20 is stably fixed in the device installation part 65. In addition, the device holder 101 includes sliders 61 for moving the device installation part 65 in the horizontal direction. The sliders 61 are arranged in both end parts of the support 55 in the leftward/rightward direction. The sliders 61 support the device installation part 65. The sliders 61 have rail shapes. Accordingly, the sliders 61 can move the device installation part 65 between the first and second positions. The first position is a position in which the device-side ports 21 and the holder-side ports 11 are aligned in the vertical direction so that the cell cultivation device is covered by holder-side lid 10 of device holder 101 (see FIGS. 5 and 6). The second position is a position in which the cell cultivation device 20 is entirely exposed without being covered by the holder-side lid 10. The leaf springs 60a (60b) are examples of a “first biasing element” in the claims.

As shown in FIG. 8, the pin hole 81a and the pin hole 81b are formed in the installation surface 56 of the device installation part 65 to receive the pins 80 (see FIG. 3).

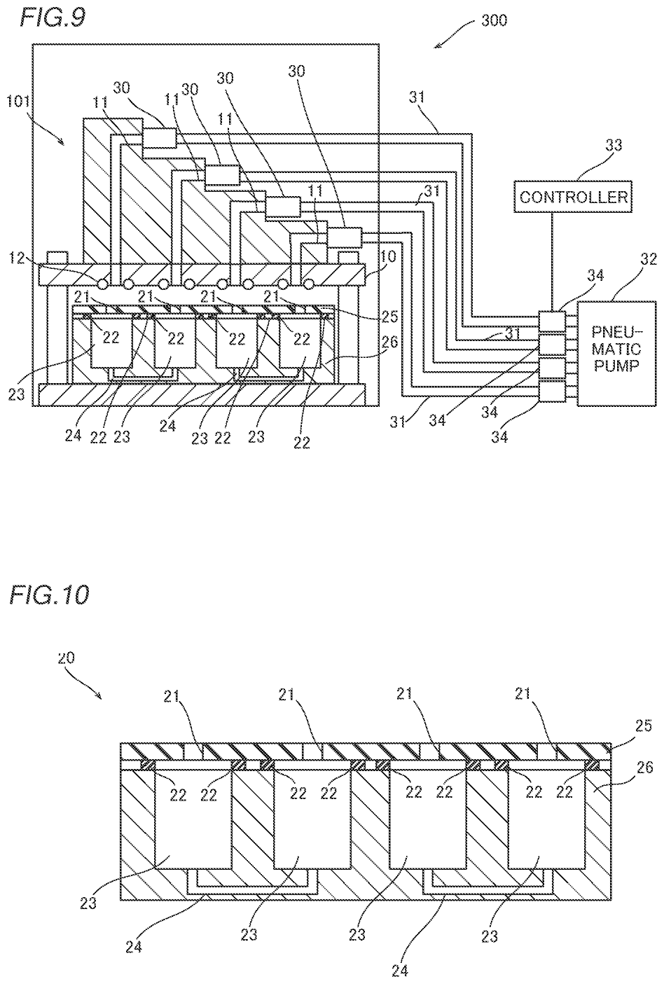

The device holder 101 is installed in an incubator 300, as shown in FIG. 9. The pneumatic pump 32, which is connected to the device holder 101 through the gas pipes 31, is located outside the incubator 300. The incubator 300 has holes corresponding to the gas pipes 31 for connecting the device holder 101 to the pneumatic pump 32.

The cell cultivation apparatus 100 includes the pneumatic pump 32, as shown in FIG. 9. In addition, the cell cultivation apparatus 100 includes the mass flow controllers 34 each of which is provided to corresponding one of the gas pipes 31 between the pneumatic pump 32 and the device holder 101. Pressures of the mass flow controllers 34 are controlled by the controller 33. Also, the controller can individually control the mass flow controllers 34 based on cell cultivation conditions. The pneumatic pump 32 is an example of a “gas supplier” in the claims.

The holder-side lid 10 includes holder-side ports 11 and O-rings 12 as shown in FIG. 9. The O-ring 12 are arranged on a lower surface of the holder-side lid 10 to surround the holder-side ports 11. The O-rings 12 seal connections between the device-side ports 21 and the holder-side ports 11 by pressing from the holder-side lid 10. The O-rings 12 are examples of “first seals” in the claims.

As shown in FIGS. 9 and 10, the cell cultivation device 20 includes a cell cultivation device body 26 and the device-side lid 25. The cell cultivation device body 26 includes cultivation chambers 23, microfluidic channels 24, and gaskets 22. The device-side lid 25 includes the device-side ports 21. The cell cultivation device 20 is arranged in the device installation part 65, and the cell cultivation device body 26 and the device-side lid 25 are pressed when the operating lever 40 is rotated in the first direction 41. In order to seal the cell cultivation device body 26 and the device-side lid 25, the gaskets 22 are arranged to be in contact with an upper surface of the cell cultivation device body 26 and a lower surface of the device-side lid 25. The gaskets 22 are examples of “second seals” in the claims.

When the cell cultivation device 20 is installed, the device installation part 65 is slid from the first position (see FIG. 1) directly below the holder-side lid 10 so as to be withdrawn to the second position (see FIG. 2). The cell cultivation device 20 is placed into the device installation part 65 withdrawn. In this installation, the leaf springs 60a and leaf springs 60b presses the cell cultivation device 20 toward the right-side wall 62a and the rear-side walls 62b, respectively. The right-side wall 62a and the rear-side walls 62b position the cell cultivation device 20 so as to position the device-side ports 21 of the cell cultivation device 20 directly below the holder-side ports 11 of the holder-side lid 10 when the device installation part 65 is in the first position. After the cell cultivation device 20 is set to the device installation part 65, the device installation part 65 is slid to the first position in which the device installation part is arranged directly below the holder-side lid 10. In a case in which the device installation part 65 is positioned in the first position, the holder-side lid 10 is positioned at a position upward of the device installation part 65 in the vertical direction. When the operating lever 40 is rotated in the first direction 41, the holder-side lid 10 is pressed toward a connection position. The connection position is a position in which the gas pipes 31 and the holder-side ports 11 are connected to the device-side ports 21 of the device-side lid 25 of the cell cultivation device 20. Also, the cam mechanism 50 presses the O-rings 12 arranged between the device-side ports 21 and the holder-side ports 11 in the connection position. Specifically, the cam mechanism 50 is configured to press the O-rings 12 against the device-side lid 25 by lowering the holder-side lid 10 by a predetermined amount from the disconnection position toward the connection position, and to compress the gaskets 22 through the device-side lid 25 by pressing the device-side lid. As a result, the cell cultivation device 20 is installed.

Also, when the cell cultivation device 20 is removed, the holder-side lid 10 is pushed up to the disconnected position by rotating the operating lever 40 in the second direction 42. The disconnection position is a position in which the device-side ports 21 are disconnected from the gas pipes 31 and the holder-side ports 11. Subsequently, the device installation part 65 is withdrawn from the first position directly below the holder-side lid 10 to the second position without being covered by the holder-side lid 10. As a result, the upper surface of the cell cultivation device 20 is entirely exposed. The cell cultivation device 20 is removed from the device installation part 65.

### Advantages of the Embodiment

In this embodiment, the following advantages are obtained.

The cell cultivation apparatus according to this embodiment includes a device holder 101 for holding a cell cultivation device 20, the device holder including cultivation chambers 23 and a device-side lid 25 having device-side ports 21 each of which is connected to corresponding one of the cultivation chambers 23; and a gas supplier including gas pipes 31 for supplying a gas to the cultivation chambers 23, wherein the device holder 101 includes a device installation part 65 in which the cell cultivation device 20 is installed, and a holder-side lid 10 arranged above the device installation part 65, and including an attachment 30 for the gas pipes 31 and holder-side ports 11, and the holder-side lid 10 is movable between a connection position and a disconnection position, the connection position being a position in which the gas pipes 31 and the holder-side ports 11 are connected to the device-side ports 21 of the device-side lid 25 of the cell cultivation device 20, the disconnection position being a position in which the gas pipes 31 and the holder-side ports 11 are disconnected from the device-side ports 21. Accordingly, the gas pipes 31 can be collectively disconnected from the cultivation device by moving the holder-side lid 10 from the connection position to the disconnection position. As a result, it is not necessary to disconnect each of the gas pipes 31 every when the cell cultivation device 20 is removed from the cell cultivation apparatus 100. Consequently, it is possible to easily remove the cell cultivation device 20 from the device holder 101 for cell cultivation. Also, the gas pipes 31 for supplying a gas can be collectively connected/disconnected to/from the cultivation device 20 by moving the holder-side lid 10 from the disconnection position to the connection position.

In this embodiment, O-rings 12 are arranged on a lower surface of the holder-side lid 10, and each of the O-rings seals a connection between corresponding one of the device-side ports 21 and corresponding one of the holder-side ports 11; and the cell cultivation apparatus further includes a press mechanism configured to press the O-rings 12, which are arranged between the device-side ports 21 and the holder-side ports 11, in the connection position. Accordingly, because the O-rings 12 seal gaps between the device-side ports 21 and the holder-side ports 11 when the device-side ports 21 are connected to the holder-side ports 11, it is possible to supply the gas to be supplied to the chambers without leakage of the gas through the gaps between the device-side ports 21 and the holder-side ports 11. Also, because the gap between device-side ports 21 and the holder-side ports 11, and a gap between the device-side lid 25 and the holder-side lid 10 can be sealed by pressing the device-side lid 25 by using the single press mechanism at the same time, it is possible to simplify a configuration of the device holder 101.

In this embodiment, gaskets 22 are arranged on a cell cultivation device body 26 to seal the device-side lid 25 and the cell cultivation device body 26; and the press mechanism is configured to press the O-rings 12 against the device-side lid 25 by lowering the holder-side lid 10 by a predetermined amount from the disconnection position to the connection position, and to compress the gaskets 22 through the device-side lid 25 by pressing the device-side lid. Accordingly, because the gaskets 22 seal the gap between the device-side lid 25 and the holder-side lid 10, it is possible to supply the gas to be supplied to the chambers without leakage of the gas through the gap between the device-side lid 25 and the holder-side lid 10. Also, because the gap between device-side ports 21 and the holder-side ports 11, and a gap between the device-side lid 25 and the holder-side lid 10 can be sealed by pressing the device-side lid 25 by using the press mechanism at the same time, it is possible to simplify a configuration of the device holder 101.

In this embodiment, the press mechanism includes a cam mechanism 50 configured to move the holder-side lid 10 between the connection position and the disconnection position, and to press the holder-side lid 10 against the device-side lid 25 in the connection position. Accordingly, it is possible to easily moving the holder-side lid 10 upward/downward while easily pressing the holder-side lid 10 toward the cell cultivation device 20 side by using the cam mechanism 50.

In this embodiment, the press mechanism is connected to the cam mechanism 50, and includes an operating lever 40 for raising/lowering the holder-side lid 10; and the press mechanism is configured to press down the holder-side lid 10 when the operating lever 40 is moved in a first direction 41 to rotate the cam mechanism 50 in a first rotation direction, and to raise the holder-side lid 10 when the operating lever 40 is moved in a second direction 42 to rotate the cam mechanism 50 in a second rotation direction. Accordingly, because the holder-side lid 10 can be raised/lowered simply by moving the operating lever 40, it is possible to easily connect the holder-side lid 10 to the device-side lid 25.

In this embodiment, the device installation part 65 is slidable in a horizontal direction between a first position in which the device-side ports 21 and the holder-side ports 11 are aligned in a vertical direction so that the cell cultivation device is covered by the holder-side lid 10 of the device holder 101, and a second position in which the cell cultivation device 20 is entirely exposed without being covered by the holder-side lid 10. Accordingly, the device installation part 65 can be slid from a position below the holder-side lid 10 to a position without being covered by the holder-side lid 10. Consequently, when the cell cultivation device 20 is removed or installed, work for cell cultivation can be conducted without interfere with the holder-side lid 10.

In this embodiment, the device installation part 65 includes a placement surface on which the cell cultivation device 20 is placed, sidewalls arranged around the placement surface, and leaf springs 60a (60b) for biasing the cell cultivation device 20 placed on the placement surface against some of the sidewalls. Accordingly, the cell cultivation device 20 is stably installed at a predetermined position without rattling when the cell cultivation device is fixed to the device installation part 65.

In this embodiment, the device holder 101 includes compression coil springs 52 for biasing the holder-side lid 10 upward toward the disconnection position. As a result, when the holder-side lid 10 is moved to the disconnection position, it is possible to support the holder-side lid without sinking to a position in which device-side lid 25 is located.

In this embodiment, the attachments 30 are arranged in a direction extending along an upper surface of the holder-side lid 10, which is included in the holder-side lid 10, for the gas pipes 31, and each of the attachments 30 for the gas pipes 31 is connected to corresponding one of the gas pipes 31. Here, in cell cultivation, the device holder 101 is placed in a thermostatic chamber such as an incubator 300 so that cells are cultivated. Because a height of the incubator 300 is limited, the device holder 101 can be more easily placed in the incubator 300 as a size of the device holder is reduced. As described above, because the gas pipes 31 can be mounted in a horizontal direction, it is possible to reduce a height of the device holder 101 as compared with a case in which the gas pipes are mounted in a vertical direction. Consequently, the device holder can be installed in a facility such the incubator 300 without interfere of the attachments 30 for the gas pipes 31 and the gas pipes 31 connected to the attachments 30 with the facility.

In this embodiment, mass flow controllers 34 each of which is provided to corresponding one of the gas pipes 31, and a controller 33 configured to individually control the mass flow controllers 34 based on cell cultivation conditions are further provided. Accordingly, gas supply pressure of the gas pipes 31 can be individually controlled by the controller 33.

### Modified Embodiments

Note that the embodiment and the example disclosed this time must be considered as illustrative in all points and not restrictive. The scope of the present invention is not shown by the above description of the embodiment and the example but by the scope of claims for patent, and all modifications (modified embodiments) within the meaning and scope equivalent to the scope of claims for patent are further included.

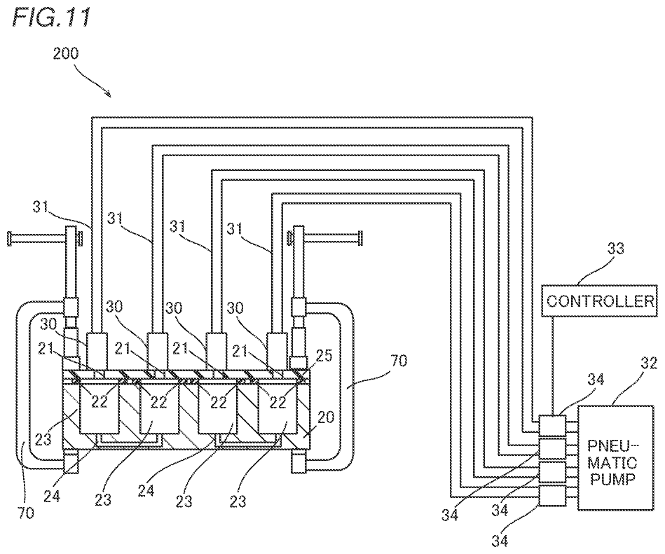

While the example in which the press mechanism is constructed of the cam mechanism 50 has been shown in the aforementioned embodiment, the present invention is not limited to this. For example, alternatively, the device-side lid 25 can be pressed by threads of clamps 70 as shown in FIG. 11.

While the example in which the lever is manually moved has been shown in the aforementioned embodiment, the present invention is not limited to this. Alternatively, an electric motor, or the like can be attached to the holder-side lid 10 or the device installation part 65 so that the lever is automatically opened/closed by a program instead of manual movement of the lever.

While the example in which the device-side lid 25 is pressed by a solid press mechanism such as lever movement has been shown in the aforementioned embodiment, the device-side lid can be pressed by applying a pressure from the top side or the bottom side by using an air pressure, or the like.

### (Modes)

The aforementioned exemplary embodiment will be understood as concrete examples of the following modes by those skilled in the art.

### (Mode Item 1)

A cell cultivation apparatus includes a device holder for holding a cell cultivation device, the cell cultivation device including cultivation chambers and a device-side lid having device-side ports each of which is connected to corresponding one of the cultivation chambers; and a gas supplier including gas pipes for supplying a gas to the cultivation chambers, wherein the device holder includes a device installation part in which the cell cultivation device is installed, and a holder-side lid arranged above the device installation part, and including an attachment for the gas pipes and holder-side ports, and the holder-side lid is movable between a connection position and a disconnection position, the connection position being a position in which the gas pipes and the holder-side ports are connected to the device-side ports of the device-side lid of the cell cultivation device, the disconnection position being a position in which the gas pipes and the holder-side ports are disconnected from the device-side ports.

### (Mode Item 2)

In the cell cultivation apparatus according to mode item 1, first seals are arranged on a lower surface of the holder-side lid, and each of the first seals seals a connection between corresponding one of the device-side ports and corresponding one of the holder-side ports; and the cell cultivation apparatus further comprises a press mechanism configured to press the first seals, which are arranged between the device-side ports and the holder-side ports, in the connection position.

### (Mode Item 3)

In the cell cultivation apparatus according to mode item 2, second seals are arranged on a cell cultivation device body to seal the device-side lid and the cell cultivation device body; and the press mechanism is configured to press the first seals against the device-side lid by lowering the holder-side lid by a predetermined amount from the disconnection position toward the connection position, and to compress the second seals through the device-side lid by pressing the device-side lid.

### (Mode Item 4)

In the cell cultivation apparatus according to mode item 2 or 3, the press mechanism includes a cam mechanism configured to move the holder-side lid between the connection position and the disconnection position, and to press the holder-side lid against the device-side lid in the connection position.

### (Mode Item 5)

In the cell cultivation apparatus according to mode item 4, the press mechanism is connected to the cam mechanism, and includes an operating lever for raising/lowering the holder-side lid; and the press mechanism is configured to press down the holder-side lid when the operating lever is moved in a first direction to rotate the cam mechanism in a first rotation direction, and to raise the holder-side lid when the operating lever is moved in a second direction to rotate the cam mechanism in a second rotation direction.

### (Mode Item 6)

In the cell cultivation apparatus according to any of mode items 1 to 5, the device installation part is slidable in a horizontal direction between a first position in which the device-side ports and the holder-side ports are aligned in a vertical direction so that the cell cultivation device is covered by the holder-side lid of the device holder, and a second position in which the cell cultivation device is entirely exposed without being covered by the holder-side lid.

### (Mode Item 7)

In the cell cultivation apparatus according to any of mode items 1 to 6, the device installation part includes a placement surface on which the cell cultivation device is placed, sidewalls arranged around the placement surface, and a first biasing element for biasing the cell cultivation device placed on the placement surface against the sidewall.

### (Mode Item 8)

In the cell cultivation apparatus according to any of mode items 1 to 7, the device holder includes a second biasing element for biasing the holder-side lid upward toward the disconnection position.

### (Mode Item 9)

In the cell cultivation apparatus according to any of mode items 1 to 8, attachments are arranged in a direction extending along an upper surface of the holder-side lid as the attachment, which is included in the holder-side lid, for the gas pipes, and each of the attachments for the gas pipes is connected to corresponding one of the gas pipes.

### (Mode Item 10)

In the cell cultivation apparatus according to any of mode items 1 to 9, pressure control mechanisms provided to corresponding one of the gas pipes; and a controller configured to individually control the pressure control mechanisms based on a cell cultivation condition specified are further provided.

### DESCRIPTION OF REFERENCE NUMERALS

10; holder-side lid
12; O-ring (first seal)
20; cell cultivation device
21; device-side port
22; gasket (second seal)
23; cultivation chamber
24; microfluidic channel
25; device-side lid
26; cell cultivation device body
30; attachment
31; gas pipe
32; pneumatic pump
33; controller
34; mass flow controller (pressure control mechanism)
40; operating lever
50; cam mechanism
51; contact plate
52; compression coil spring (second biasing element)
60a, 60b; leaf spring (first biasing element)
61; slider
62a, 62b, 62c, 62d; sidewall
65; device installation part
100; cell cultivation apparatus
101; device holder
300; incubator

1. A cell cultivation apparatus comprising:
a device holder for holding a cell cultivation device, the cell cultivation device including cultivation chambers and a device-side lid having device-side ports each of which is connected to corresponding one of the cultivation chambers; and
a gas supplier including gas pipes for supplying a gas to the cultivation chambers, wherein
the device holder includes a device installation part in which the cell cultivation device is installed, and
a holder-side lid arranged above the device installation part, and including an attachment for the gas pipes and holder-side ports, and
the holder-side lid is movable between a connection position and a disconnection position, the connection position being a position in which the gas pipes and the holder-side ports are connected to the device-side ports of the device-side lid of the cell cultivation device, the disconnection position being a position in which the gas pipes and the holder-side ports are disconnected from the device-side ports.

2. The cell cultivation apparatus according to claim 1, wherein
first seals are arranged on a lower surface of the holder-side lid, and each of the first seals seals a connection between corresponding one of the device-side ports and corresponding one of the holder-side ports; and
the cell cultivation apparatus further comprises a press mechanism configured to press the first seals, which are arranged between the device-side ports and the holder-side ports, in the connection position.

3. The cell cultivation apparatus according to claim 2, wherein
second seals are arranged on a cell cultivation device body to seal the device-side lid and the cell cultivation device body; and
the press mechanism is configured to press the first seals against the device-side lid by lowering the holder-side lid by a predetermined amount from the disconnection position toward the connection position, and to compress the second seals through the device-side lid by pressing the device-side lid.

4. The cell cultivation apparatus according to claim 2, wherein the press mechanism includes a cam mechanism configured to move the holder-side lid between the connection position and the disconnection position, and to press the holder-side lid against the device-side lid in the connection position.

5. The cell cultivation apparatus according to claim 4, wherein
the press mechanism is connected to the cam mechanism, and includes an operating lever for raising/lowering the holder-side lid; and
the press mechanism is configured to press down the holder-side lid when the operating lever is moved in a first direction to rotate the cam mechanism in a first rotation direction, and to raise the holder-side lid when the operating lever is moved in a second direction to rotate the cam mechanism in a second rotation direction.

6. The cell cultivation apparatus according to claim 1, wherein the device installation part is slidable in a horizontal direction between a first position in which the device-side ports and the holder-side ports are aligned in a vertical direction so that the cell cultivation device is covered by the holder-side lid of the device holder, and a second position in which the cell cultivation device is entirely exposed without being covered by the holder-side lid.

7. The cell cultivation apparatus according to claim 1, wherein the device installation part includes a placement surface on which the cell cultivation device is placed, sidewalls arranged around the placement surface, and a first biasing element for biasing the cell cultivation device placed on the placement surface against the sidewall.

8. The cell cultivation apparatus according to claim 1, wherein the device holder includes a second biasing element for biasing the holder-side lid upward toward the disconnection position.

9. The cell cultivation apparatus according to claim 1, wherein attachments are arranged in a direction extending along an upper surface of the holder-side lid as the attachment, which is included in the holder-side lid, for the gas pipes, and each of the attachments for the gas pipes is connected to corresponding one of the gas pipes.

10. The cell cultivation apparatus according to claim 1 further comprising
pressure control mechanisms provided to corresponding one of the gas pipes; and
a controller configured to individually control the pressure control mechanisms based on a cell cultivation condition specified.
